(19) United States
(12) Patent Application Publication
(10) Pub. No.: US 2024/0424195 A1
(43) Pub. Date: Dec. 26, 2024

(54) AUTOMATED ECHOCARDIOGRAM PROCESSING TO CHARACTERIZE SHUNTS

(71) Applicant: Agitated Solutions Inc., St Paul, MN (US)

(72) Inventors: Morgan Evans, Apple Valley, MN (US); Micah Eimer, Glenview, IL (US)

(73) Assignee: Agitated Solutions Inc., St Paul, MN (US)

(21) Appl. No.: 18/826,131

(22) Filed: Sep. 5, 2024

Related U.S. Application Data
(63) Continuation-in-part of application No. 18/131,349, filed on Apr. 5, 2023.
Application No. 18/131,349 is a continuation-in-part of application No. 17/321,957, filed on May 17, 2021, now Pat. No. 11,986,342.
(60) Provisional application No. 63/536,558, filed on Sep. 5, 2023.
(60) Provisional application No. 63/327,738, filed on Apr. 5, 2022.
(60) Provisional application No. 63/026,177, filed on May 18, 2020.

(51) Int. Cl. A61M 5/00; A61B 8/06; A61B 8/08; A61B 17/00

(52) U.S. Cl. CPC A61M 5/00; A61B 8/06; A61B 8/0833; A61B 8/0883; A61B 17/0057; A61B 2017/00575

(57) ABSTRACT

A method may include intravenously directing first bubbles into a patient's venous circulatory system and to a right side of the patient's heart. The first bubbles may have sizes that fall within a first range. The method may further include monitoring the patient's heart, with ultrasound imaging, to detect presence of the first bubbles on a left side of the patient's heart. Upon detecting the first bubbles on the left side of the patient's heart, the method may further include intravenously directing second bubbles into the patient's venous circulatory system and to the right side of the patient's heart. The second bubbles may have sizes that fall within a second range that is different than the first range. Upon detecting the second bubbles on the left side of the patient's heart, the method may further include initiating treatment to minimize risk of stroke in the patient.

### CROSS-REFERENCE TO RELATED APPLICATIONS

This application is a continuation-in-part of U.S. application Ser. No. 18/131,349, titled “Automated Echocardiogram Processing to Characterize Shunts,” filed Apr. 5, 2023, which claims the benefit of U.S. Provisional Application Ser. No. 63/327,738, titled “Image Processing for Assessing Shunts,” filed Apr. 5, 2022 and which is a continuation-in-part of U.S. application Ser. No. 17/321,957, titled “Characterization of Cardiac Shunts with Bubbles,” filed May 17, 2021, now U.S. Pat. No. 11,986,342, which claims the benefit of U.S. Provisional Application Ser. No. 63/026,177, titled “Characterization of Cardiac Shunts with Bubbles,” filed May 18, 2020. This application further claims priority to U.S. Provisional Application Ser. No. 63/536,558, titled “Automated Echocardiogram Processing to Characterize Shunts,” filed Sep. 5, 2023. This application incorporates the entire contents of the foregoing applications herein by reference.

### TECHNICAL FIELD

Various embodiments relate generally to diagnosing and characterizing cardiac shunts.

### BACKGROUND

A patent foramen ovale (PFO) or atrial septal defect (ASD) may be present in the septum that would otherwise separate two sides of a patient's heart. When present, either a PFO or an ASD can allow embolic material—which would otherwise be filtered out by the patient's lungs but for the PFO or ASD—to enter arterial circulation.

Paradoxical embolization through a patent foramen ovale (PFO) may be implicated in cryptogenic strokes when there is a prevalent right-to-left shunt. The causal relationship between PFOs and a stroke is controversial; although the prevalence of PFOs in the adult population is high (25% of adults in the US), presence of a PFO is not indicative of an increased risk of stroke. However, patients with a prior cryptogenic stroke have a significantly higher presence of PFOs (68%).

Patients may benefit from interventions including endovascular or surgical closure, anticoagulation, or antiplatelet therapy. Device PFO closure has found to have recurrent stroke in 0-5% of patients, major procedural complications in 1.5% of patients, and minor complications in 7.9% of patients.

Evaluating the risk-benefit of PFO treatment is difficult and wrought with anxiety for patient and provider. Between the commonality of PFOs, the procedural risk of closure, and the severity and high incidence of strokes, there is currently no scientific consensus on identifying patients in which PFO closure would be beneficial from those where it would be detrimental. Guidance on when to pursue these interventions is lacking due to diagnostic limitations for grading whether the PFO had or will have a causal role in stroke.

Transesophageal or transthoracic echocardiography (TEE or TTE) imaging with agitated saline bubbles as a contrast is the gold standard test for evaluating the presence of PFOs following stroke. Bubble studies use cardiac cycle timing of bubbles appearing from the right (RA) to left atrium (LA) to distinguish intracardiac abnormalities such as PFO from transpulmonary shunts (TPS). Three-dimensional TEE can provide some morphological features of the PFO, including tunnel length, margins or rims, and anatomical detail of surrounding structures such as the aorta and inferior vena cava. Despite the wealth of information provided by TEE, it is not readily implemented in diagnosis of PFO-associated strokes given (1) acquisition difficulties and (2) procedural constraints. Acquisition difficulties arise due to how contrast enhancement is delivered in bubble studies in standard of care (SOC), which uses the Tessari's method to agitate air and fluid (typically saline or glucose) and manual injections.

### BRIEF DESCRIPTION OF THE DRAWINGS

FIG. 1 illustrates a portion of a human circulatory system.

FIG. 2A illustrates detail of a human heart.

FIG. 2B depicts a ventricular systole phase of a heart.

FIG. 2C depicts an atrial systole phase of a heart.

FIGS. 3A-3C depict bubble studies conducted with bubbles of varying sizes.

FIG. 4 illustrates a method for conducting multiple bubble studies in sequence to characterize a cardiac shunt.

FIG. 5A illustrates an exemplary ECG waveform for a patient undergoing a bubble study.

FIGS. 5B-1 and 5B-2 illustrate echocardiogram images corresponding to the ECG of FIG. 5A.

FIG. 5C depicts a process for identifying regions of interest in echocardiogram images.

FIGS. 5D-1 and 5D-2 depict a process for determining average boundaries for exemplary regions of interest.

FIGS. 5E-1 and 5E-2 depict a method of applying average boundaries to echocardiogram images, to facilitate further analysis.

FIG. 5F depicts an exemplary method for segmenting a right atrium image and analyzing to confirm sufficient opacification during a bubble study.

FIGS. 5G-1 and 5G-2 depict methods for analyzing the left side of a patient's heart during a bubble study to detect migration of bubbles through a shunt and to characterize the shunt.

FIG. 6A-1 illustrates an exemplary ECG waveform for a patient undergoing a bubble study.

FIG. 6A-2 illustrates echocardiogram images corresponding to the ECG of FIG. 6A-1.

FIG. 6A-3 depicts a process for identifying a region of interest in echocardiogram images shown in FIG. 6A-2.

FIG. 6B-1 illustrates an exemplary ECG waveform of a patient undergoing a bubble study, while performing a Valsalva maneuver.

FIG. 6B-2 illustrates echocardiogram images corresponding to the ECG of FIG. 6B-1.

FIG. 6B-3 depicts a process for identifying a region of interest in echocardiogram images shown in FIG. 6B-2.

FIG. 6C depicts comparison of average regions of interest between a bubble study without a Valsalva maneuver being performed, and a bubble study with a Valsalva maneuver being performed.

FIG. 7 depicts an exemplary architecture for an AI-based analysis system.

FIG. 8 illustrates an exemplary interface for an AI-based analysis system.

FIG. 9 illustrates exemplary opacification and heatmap data.

### DETAILED DESCRIPTION

Agitated saline contrast studies are a useful adjunct to many ultrasound examinations, particularly cardiac ultrasound (echocardiography). Injection of agitated saline into a vein combined with echocardiography may be used to detect shunts which may be within the heart, such as a patent foramen ovale (PFO) or an atrial septal defect (ASD) (two types of holes in the heart), or external to the heart (e.g., in the lungs) known as pulmonary arteriovenous malformations (pAVM). Agitated saline can also be used with echocardiography to confirm catheter placement in fluid around the heart (pericardiocentesis), detect anomalous connections within the heart, visualize the right side of the heart, and accentuate right-sided blood flow for the purpose of quantitation.

Agitated saline contrast echocardiography takes advantage of the increased reflection that results when ultrasound waves meet a liquid/gas interface. This allows for visualization of otherwise poorly reflective areas such as fluid filled cavities by ultrasound imaging equipment. Applications in which this has been clinically useful include echocardiography where agitated saline can be used to define the structural integrity of the interatrial septum or infer the presence of a transpulmonary shunt. Agitated saline can also be combined with Doppler echocardiography to assess blood flow through the tricuspid valve. An alternative method to detect atrial defects uses ultrasound of the brain vessels (transcranial Doppler) to detect bubbles that have crossed from the right heart to the left heart and entered the cerebral circulation.

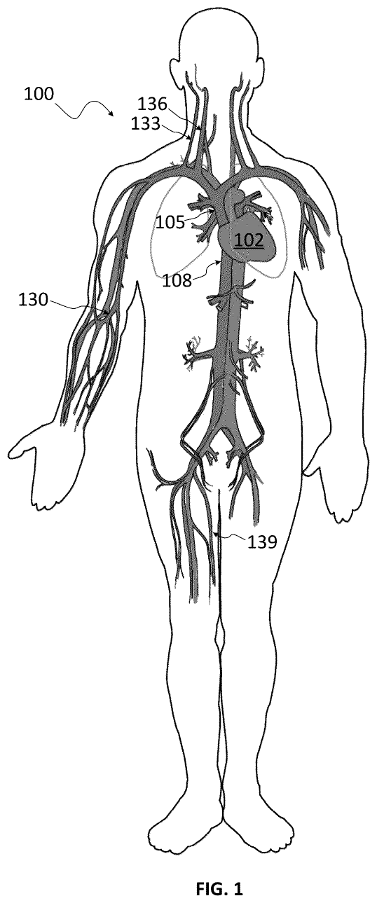

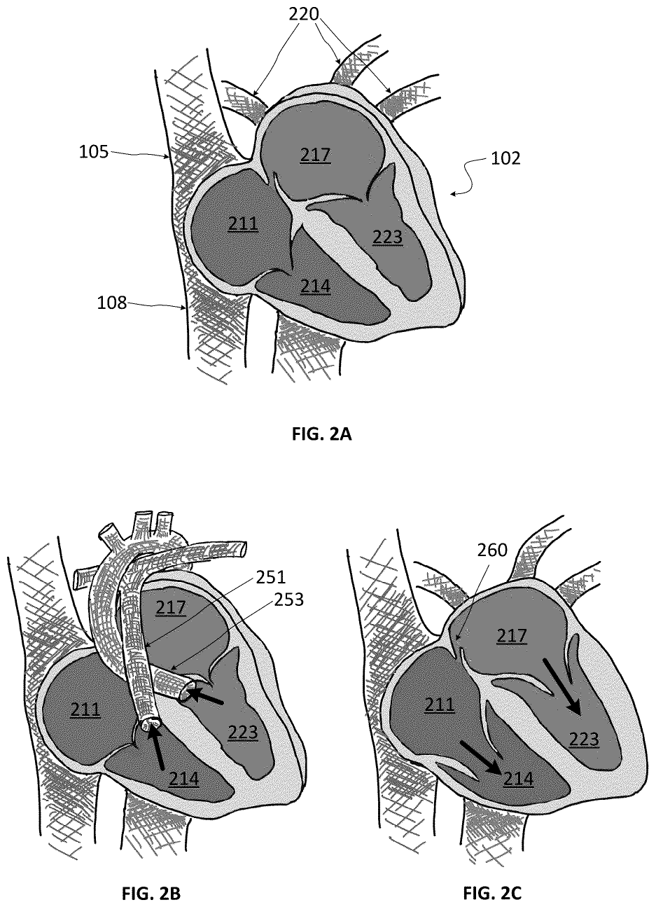

Described herein are methods for characterizing the extent of shunts, such as interatrial shunts. For context, various aspects of a human cardiovascular system are first described with reference to FIG. 1 and FIGS. 2A, 2B and 2C. FIG. 1 illustrates a portion of an overall human circulatory system 100. At its core, is the heart 102, and a system of arteries that extend from the heart, and veins that return to the heart.

Various internal structures of the heart 102 are described in greater detail with reference to FIG. 2A. Blood is returned to the heart 102 from throughout the body by the vena cava, which is divided into the superior vena cava 105, which collects blood from the upper portion of the body, and the inferior vena cava 108, which collects blood from the lower portion of the body. Blood flows through the superior vena cava 105 and inferior cava 108 on its way to the right atrium 211.

After being oxygenated in the lungs, blood is returned to the left atrium 217 of the heart 102 via the pulmonary veins 220 (three of four of which are shown). From the left atrium 217, the heart 102 pumps blood into the left ventricle 223, which in turns pumps it to the aorta for distribution throughout the body.

In more detail, with reference to FIG. 2B, the right ventricle 214 and left ventricle 223 contract during a ventricular systole phase, pumping blood from the right ventricle 214 into the pulmonary artery 251 and from the left ventricle 223 to the aorta 253. With reference to FIG. 2C, the left atrium 211 and right atrium 217 contract during an atrial systole phase, pumping blood from the right atrium 211 to the right ventricle 214 and from the left atrium 217 to the left ventricle 223. In FIG. 2C, the pulmonary artery 251 and aorta 253 have been removed to better illustrate intracardiac flow.

Also illustrated in FIG. 2C is a shunt 260 that fluidly couples the right atrium 211 and the left atrium 217. When present, such a shunt can increase a risk of stroke by allowing embolic material (e.g., a blood clot, a fat globule, other foreign material) to move from the right atrium 211 to the left atrium 217, then into the left ventricle 223, and finally into arterial circulation through the body via the aorta 253—where the embolic material can cause a stroke or other critical blockage. But for the presence of such a shunt 260, the embolic material would, in most patients, be filtered by the lungs.

Shunts may take different forms. For example, a PFO is a small flap-like opening that is normally present at birth in the heart wall (septum) that separates the left atrium from the right atrium. In some patients, this PFO never fully closes after birth. An ASD is a type of birth defect in which a hole exists in the septum dividing the atria. Often, an ASD is more serious than a PFO. Not illustrated, but also possible, is a ventricular septal defect (VSD), in which a hole exists in the septum that separates the right ventricle from the left ventricle.

When a shunt 260 is present in a patient's heart, the size of the shunt 260 may determine the risk of a traumatic or catastrophic effect of embolic material entering arterial circulation via the left atrium 217, left ventricle 223 and aorta 253, rather than being filtered out in the lungs. A larger shunt may, depending on other aspects of the patient's anatomy and physiology, allow larger emboli to enter arterial circulation, where such emboli could cause a stroke, heart attack or other traumatic blockage.

Many shunts can be treated-either surgically (e.g., with percutaneous closure using a catheter, or with open-heart surgery and direct surgical closure), or with medication (e.g., blood thinners, to reduce the risk of clots). However, many shunts need not be treated. That is, many shunts are small enough, and patients may be otherwise healthy enough that the reduction of risk by closing a small shunt may be outweighed by the risk of a procedure to close the shunt.

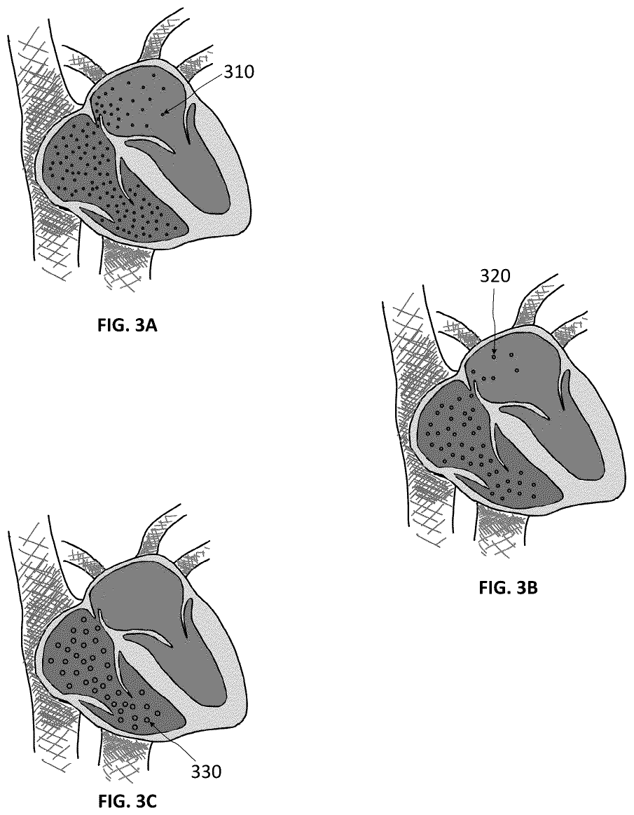

For example, in some patients with ASDs, where the ASD is less than 5 mm and there is no evidence of either right ventricular volume overload or paradoxical embolism, it may be safer to not surgically treat the ASD; on the other hand, ASDs that are larger than 5 mm or in patients where right ventricle overload is detected, or where the patient has suffered a cryptogenic stroke, surgical repair of the ASD may be indicated. Similarly, in some patients, surgical repair may be indicated for PFOs that exceed 4 mm in diameter, or in patients that have recently suffered a cryptogenic stroke. For these reasons, it can be advantageous to accurately assess the size of the shunt, to facilitate weighing of risks between repairing an ASD or PFO or leaving it untreated surgically.

Whereas a single bubble study may be used to assess the presence of a cardiac shunt, additional diagnostic information can be obtained from multiple bubble studies, especially when it is possible to control for bubble size and progressively and predictably increase the size of bubbles from one bubble study to the next. A method for assessing size of a cardiac shunt is now described with reference to FIG. 3A, FIG. 3B and FIG. 3C.

As depicted in FIG. 3A, a bubble study may be conducted using small bubbles 310 (e.g., bubbles having a diameter in the approximate range of 8 um to 15 um, where “approximate” may mean within 1%, 5%, 10%, 20%, 25% or 50% of a nominal value). Upon confirmation of a shunt (e.g., by detection of immediate (e.g., within one, two or three atrial systole phases) bubble migration from right atrium to left atrium, a second bubble study may be conducted using larger bubbles 320 (e.g., bubbles having a diameter in the approximate range of 15 um to 25 um), as depicted in FIG. 3B. In some cases, regardless of the outcome the second bubble study, a third bubble study, using still larger bubbles 330 (e.g., bubbles having a diameter in the approximate range of 25 um to 35 um), as depicted in FIG. 3C, may be conducted; in other cases, the third bubble study may only be conducted if a shunt is confirmed by the second bubble study.

Described above is a sequence of bubble studies in which the first study is with “small” bubbles and the last study is conducted with “large” bubbles. This order may also be reversed. For example, in some patients where a cardiac shunt is anticipated (e.g., following a cryptogenic stroke), a “large” bubble study may be conducted first. In such a case, if a suspected cardiac shunt is confirmed, no additional bubble studies may be required. Three bubble studies are described above, but in some cases, even when the first study involves small bubbles, only two bubble studies may be required. In some cases, multiple (e.g., two, three, four or more) bubble studies may be conducted using the same size bubbles. In some cases, different size bubbles may be used; for example, when the risk of inducing an air embolism is determined to be low for a specific patient, bubbles larger than 35 um may be employed. The methods described herein may be modified in order, repetition and in other ways, at the discretion of the medical care provider.

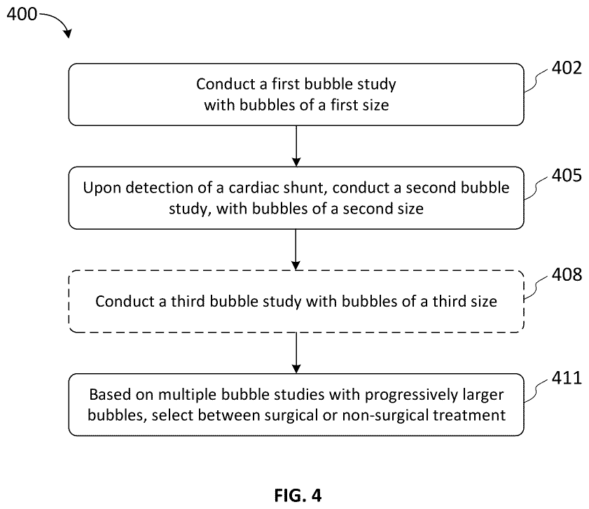

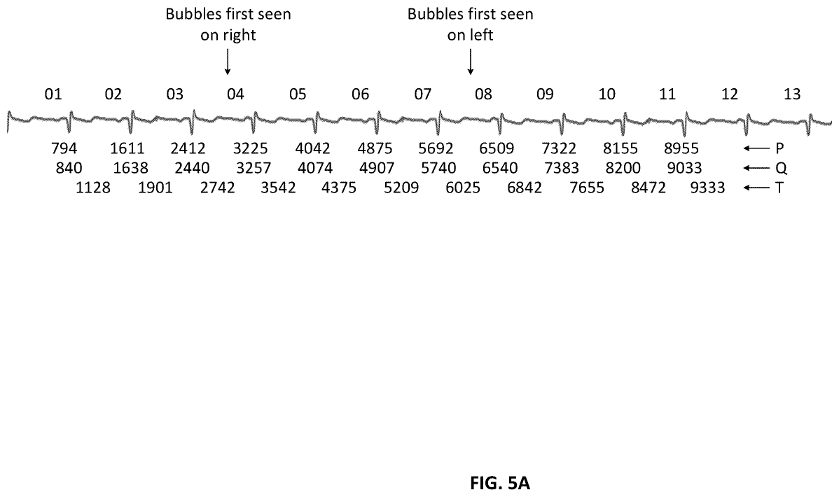

FIG. 4 illustrates a method 400 for conducting a sequence of bubble studies. The method 400 includes conducting (402) a first bubble study with bubbles of a first size. For example, a bubble study with small bubbles 310, such as depicted in FIG. 3A, may be first conducted (402) to identify a shunt of any significant size. The first bubble study may be motivated by a stroke (e.g., a cryptogenic stroke, or stroke of unknown causes), or it may be part of a cardiac workup that is motivated by other reasons.

When a shunt is detected, for example by detection of bubbles on the left side of the heart (either in the left atrium or left ventricle) within one systole phase of their presence on the right side of the heart (or, in some implementations, within two or three atrial systole phases), the method 400 includes conducting (405) a second bubble study with bubbles of a second size. For example, a bubble study with larger bubbles 320, as depicted in FIG. 3B, may be conducted (405). Detection of these larger bubbles 320 on the left side of the heart can provide further information about the nature and extent of the shunt.

Optionally (and as a matter of course, in some cases, when bubbles in the second bubble study are detected on the left side of the heart), a third bubble study may be conducted (408) with still-larger bubbles. For example, the third bubble study may employ still-larger bubbles 330.

Based on the multiple studies with progressively larger bubbles, it may be possible to characterize a size or extent of a cardiac shunt; based on this characterization, the method 400 can include selecting (411) between surgical or non-surgical treatment of the shunt.

For example, if a first bubble study with small bubbles, such as the study depicted in FIG. 3A, identifies a shunt in a patient's heart, but bubbles are not detected on the left side of the patient's heart in a follow-on study with larger bubbles, and the patient has no other underlying health concerns that would make an unclosed shunt particularly high-risk, the shunt may be left untreated surgically. In some cases, the patient may be provided with a blood thinner, to reduce the risk of clots, or the patient may be provided with some other prophylactic medication. As another example, if bubble migration from right to left were detected in a patient in three bubble studies with progressively larger bubbles, it may be determined that a shunt of significant size is present, and surgical closure may be indicated.

By controlling the size of the bubbles (e.g., by generating them in a manner that results in relatively consistent and fixed sizing—for example, one in which a median bubble size may fall within a specified range, or in which a majority of bubbles within a distribution of bubble sizes may fall within one, two or three standard deviations of a specified range) and by measuring or inferring other cardiac parameters, such as blood volume and blood flow, it may be possible to characterize cardiac shunts in far greater detail than would otherwise be possible. In addition to detecting mere presence of bubbles of any given size, a volume or number of bubbles may also be measured. In this manner, particularly when bubble volume, blood volume and blood flow are all measured or inferred, it may be possible to characterize larger shunts, even if it is not possible (e.g., for safety reasons) to use very large bubbles alone to character such shunts.

Multiple techniques may be employed to detect the presence of bubbles on the left side of the heart. For example, a noninvasive transthoracic echocardiogram (TTE) may be employed, whereby an ultrasound transducer is placed on the chest of a patient undergoing the bubble study. High frequency soundwaves (ultrasound) are used to create a moving picture of the heart, through the chest wall, and when the ultrasound and bubble study are properly performed, bubbles that are present on either side of the heart will be picked up and imaged through the procedure.

A transesophageal echocardiogram (TEE) may also be employed for higher resolution of images. In a TEE, and ultrasound transducer is placed in the esophagus of the patient undergoing the procedure. Given the proximity of the esophagus to the heart, and given that the ultrasound in a TEE does not have to traverse the chest wall and rib cage, the images from a TEE are typically much clearer than with a TTE.

Machine learning algorithms may be employed across multiple bubble studies to provide additional diagnostic information. For example, precise measurements could be captured from the ultrasound images in ether TTE or TEE procedures, to determine blood volume in each chamber of a patient's heart. Individual bubbles could be traced to capture a blood flow rate. Regression analysis could be applied across many patients to determine likelihood of bubbles (or bubbles of a particular size) appearing on the left side of the patient's heart when a shunt is present, or a shunt of a particular kind, given specific volumes or flow rates. When such a shunt is identified and repaired, more precise information about its size could be gleaned during the procedure for its repair, and this information could be fed back into the machine learning algorithm. Other variables could be incorporated into such machine learning algorithms (e.g., patient age, other heart or general health conditions, respiratory function, gender, genetics, blood type, etc.).

To facilitate bubble studies or other diagnostic or therapeutic procedures whereby bubbles are to be introduced into the circulatory system, one must get the bubbles into the venous system and ultimately into the superior vena cava 105 or inferior vena cava 108, and into the right atrium 211 of the heart 102. With reference to FIG. 1, there are several common access points through which bubbles can be so introduced. Common among them is intravenous introduction of bubbles (e.g., mixed with saline or dextrose) via the median cubital vein 130 of the right arm. From here, blood flows through the basilic vein, axillary vein, subclavian vein, and into the superior vena cava 105. Alternative paths to the superior vena cava 105 are the external jugular vein 133, or internal jugular vein 136, both of which drain into the brachiocephalic vein prior to reaching the superior vena cava 105. An alternative inferior route includes the femoral vein 139, which flows into the inferior vena cava 108 prior to reaching the right atrium 211. Other routes to the right atrium 211 are possible.

### Image Processing Using Artificial Intelligence/Machine Learning

Instead of cardiologists counting microbubbles, the culpability of thrombus embolization through a PFO could instead theoretically be quantified by the time-course of contrast enhancement in the LA/RA with artificial intelligence (AI).

Application of AI to echocardiography broke ground in early 2020, when the FDA authorized Caption Guidance software which collects cardiac US data for tasks including classification of standard US videos, automated segmentation, left ventricular volume calculations, and diagnosis. The intention behind utilizing AI in US is to automate otherwise complex processes that require significant time and thus cost from echocardiographers, as well as reduce user subjectivity for more accurate and standardized analysis. Deep learning models are a growing subfield which use layers of algorithms within an artificial neural network to enable the AI software to learn input/output translation without supervision. Using TEE US microbubble studies as an input into a machine learning algorithm that outputs PFO or intra-pulmonary shunt (IPS) detection, as well as an ordinal score of PFO risk for recurrent stroke, would revolutionize evidence-based practice in PFO assessment. Outlined herein is a process for applying machine learning to improve the diagnostic value of bubble studies.

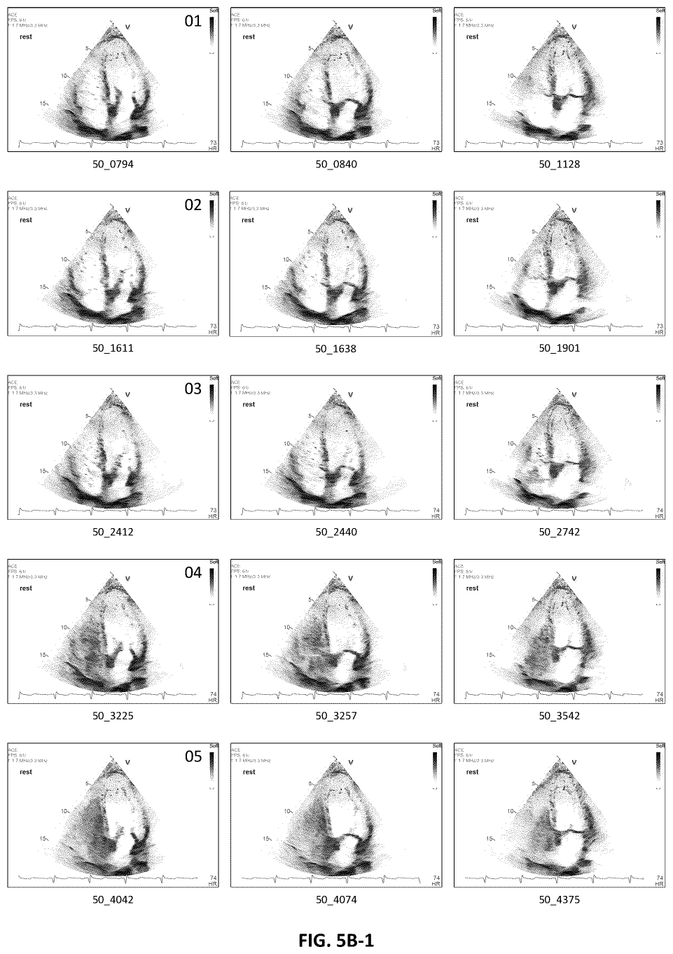

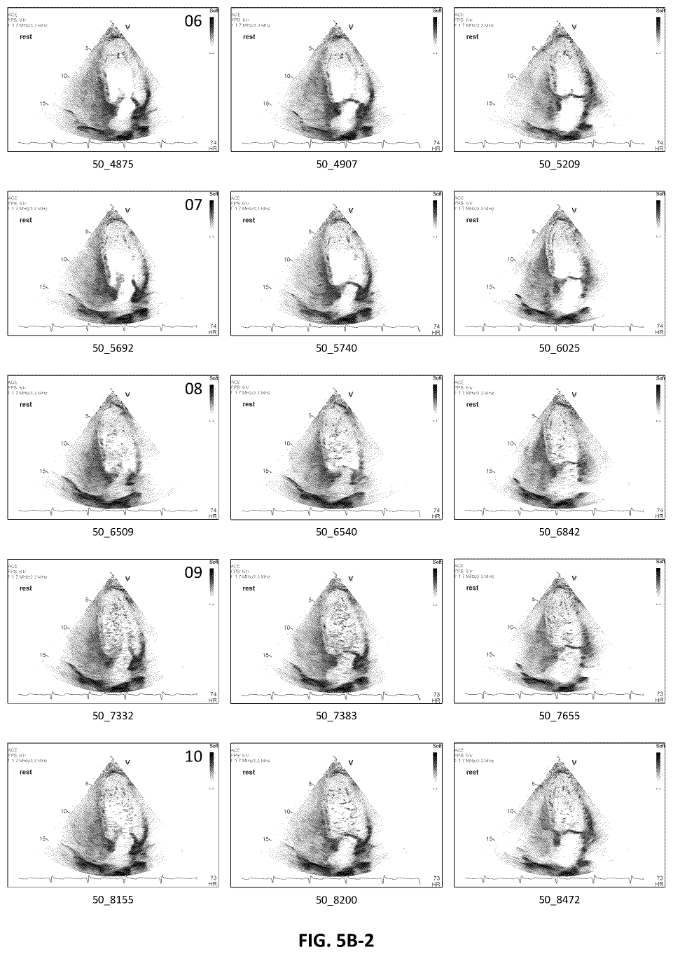

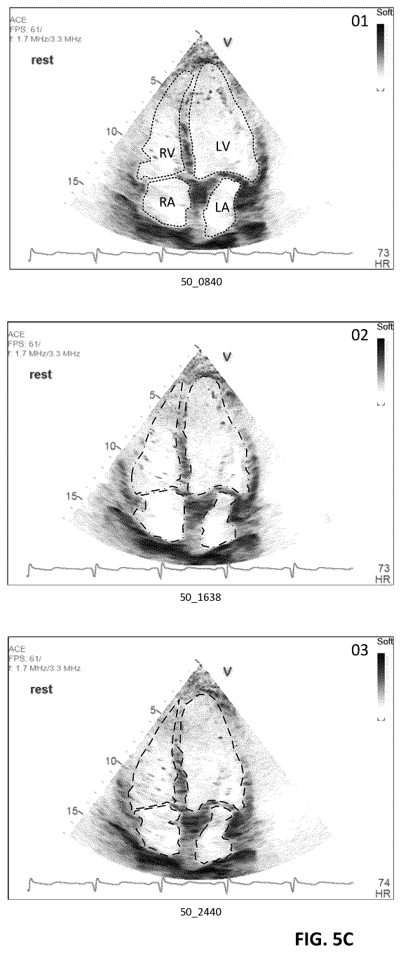

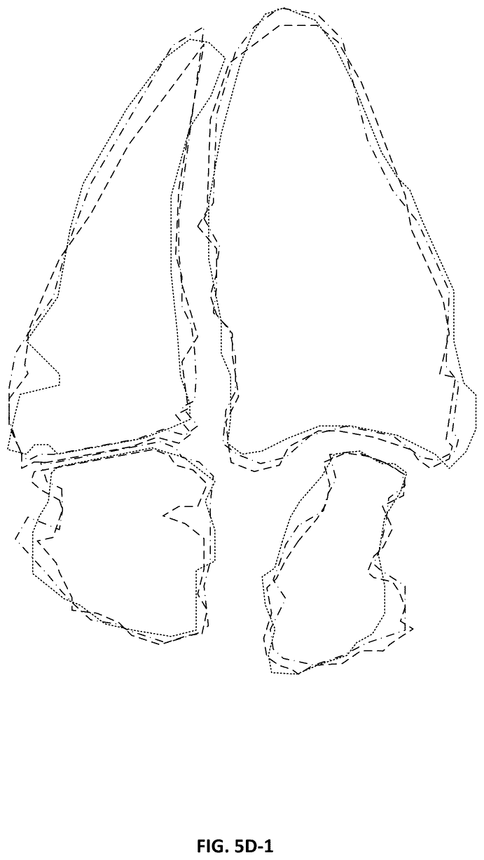

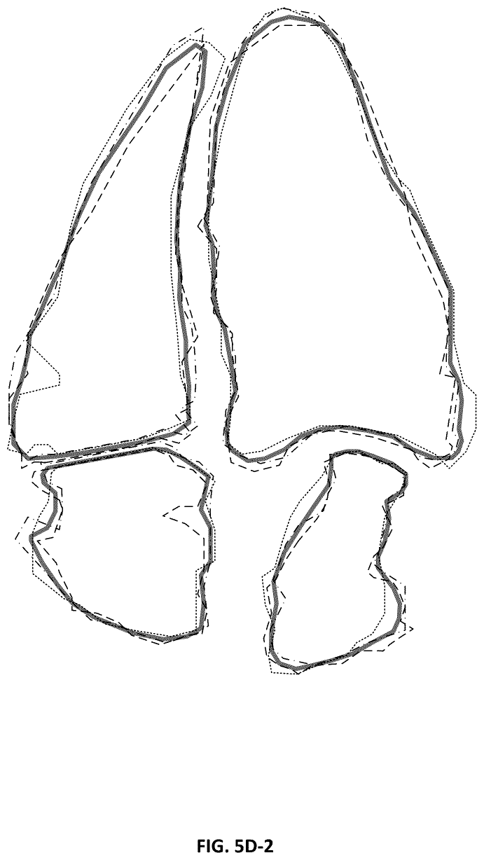

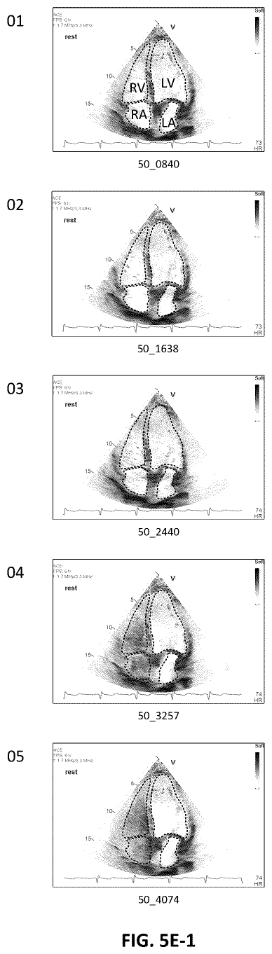

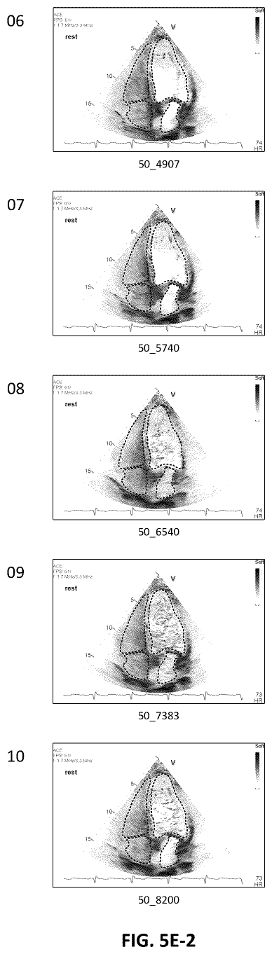

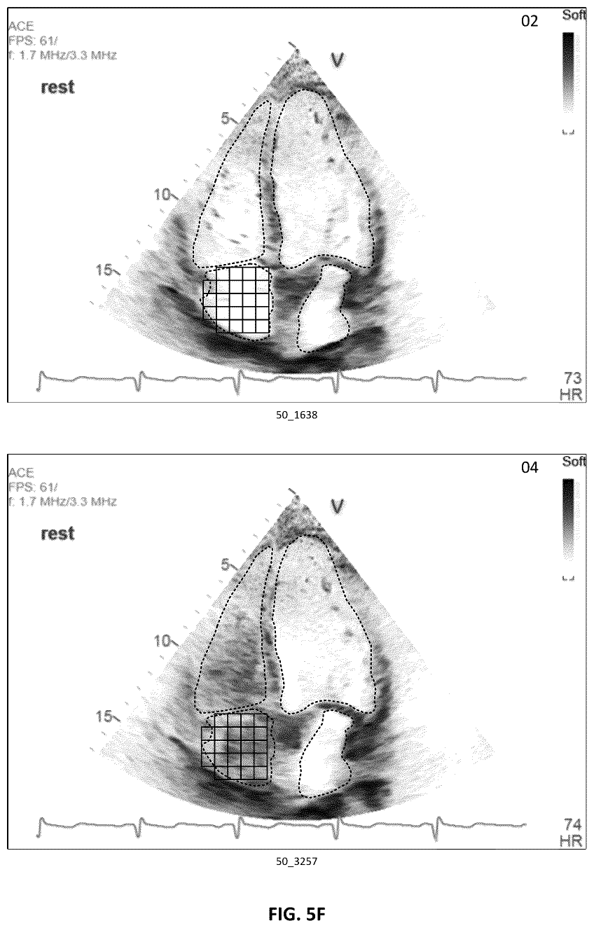

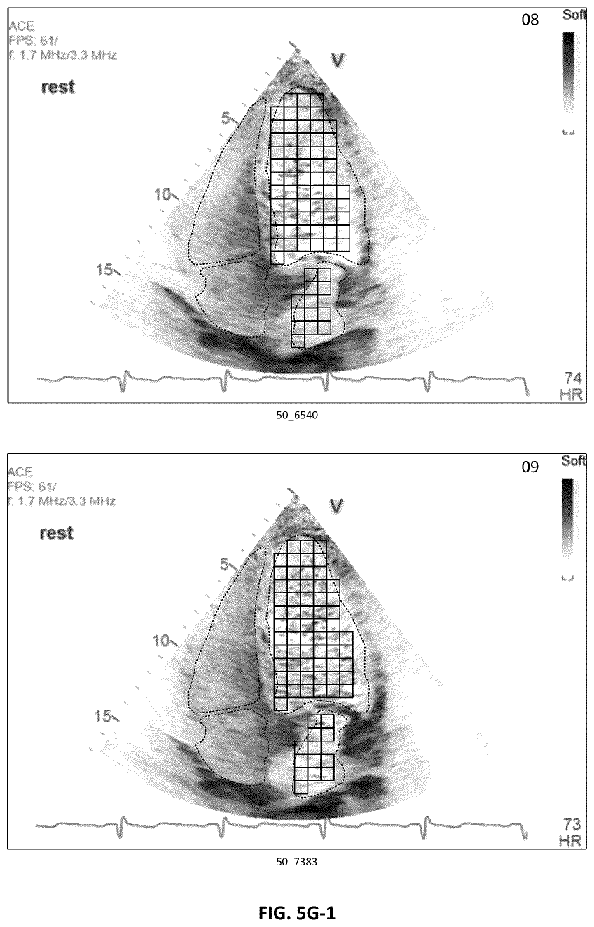

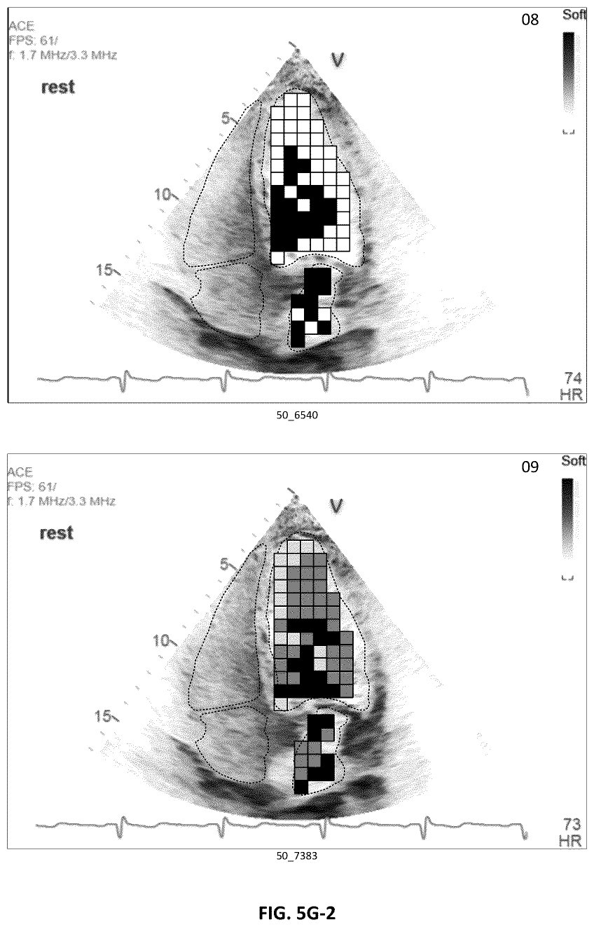

FIG. 5A illustrates an ECG waveform that corresponds to a sequence of ultrasound images from a patient (“patient 50” in one study of fifty echocardiograms) that will be illustrated and described with reference to figures that follow. As illustrated, the ECG waveform comprises 13 cardiac cycles (labeled). Timestamps that will be referenced in following figures are provided—in particular, time stamps associated with the start of the P wave, the start of the Q wave, and the start of the T wave.

FIGS. 5B-1 and 5B-2 illustrate ultrasound images corresponding to ten of the cardiac cycles captured in the ECG waveform of FIG. 5A. For clarity, as illustrated, black and white have been inverted relative to a standard ultrasound image—that is, ultrasound images are illustrated herein as black on white, rather than the standard white on black ultrasound image. Individual image labels in FIGS. 5B-1 and 5B-2 correspond to the time stamps shown in FIG. 5A. The ultrasound images were taken during a bubble study of “patient 50” using a TTE procedure. As indicated in FIG. 5A, microbubbles were injected into patient 50, and these bubbles were first seen on the right side of the patient's heart during cycle 04; bubbles were seen on the left side of the patient's heart during cycle 07—indicating a shunt in the patient's heart.

In the images shown in FIGS. 5B-1 and 5B-2, bubbles are visible to a human eye on the right side of the patient's heart. In some implementations, the presence of such bubbles can be detected automatically, for example, through an exemplary method that is now described.

FIG. 5C illustrates three snapshots in time, corresponding to substantially the same point in three different cardiac cycles (e.g., as shown, approximately at the end of the QRS phase). (As used herein, “substantially” or “about” or “approximately” may refer to within 1% or 5% or 10% or 25% of a nominal value; with respect to cardiac cycles, the same terms may refer to very near to an identified portion of a phase, when considered relative to the time period(s) of that phase and adjacent phases—for example, “approximately at the end of the QRS phase” may refer to the last 1-25% of the QRS phase or the first 1-5% of the ST phase.)

Multiple images corresponding to the same point of different cardiac cycles may be analyzed to delineate different structures of the heart. FIG. 5C illustrates outlines corresponding to the four chambers of a human heart—the right ventricle (“RV”), left ventricle (“LV”), left atrium (“LA”) and right atrium (“RA”). These chambers may be identified through image analysis of each ultrasound snapshot. That is, in some implementations, a grayscale or color value for each pixel or cluster of pixels may be analyzed relative to adjacent pixels; and any pixel or cluster of pixels that is lighter than (or darker than) at least one adjacent pixel or cluster of pixels (e.g., by some threshold value) may be identified as corresponding to a cardiac chamber, rather than cardiac tissue. Boundaries may then be identified for each chamber, according to analysis of a given ultrasound image—as depicted in FIG. 5C.

Boundaries from different cardiac cycles may be averaged together—as depicted in FIGS. 5D-1 and 5D-2. Average boundaries may then be superimposed on the original images to facilitate further analysis—as depicted in FIGS. 5E-1 and 5E2.

Described herein is pixel-by-pixel analysis that compares grayscale or color values between pixels and relative to a threshold value. The threshold value may vary based on location within the heart. For example, some ultrasound images may capture the atria more clearly than the ventricles; thus, threshold values used in delineating chambers from tissue may be different for the atria than for the ventricles. In some implementations, the threshold may vary across a given chamber. For example, for images such as those shown in FIGS. 5B-1 and 5B-2—where the atria and the portion of the ventricles that are closest to the atria are clearer than the distal portions of the ventricles-thresholds may vary from one end of the heart to the other. Moreover, thresholds may be determined for each patient, based, for example, on specific color or grayscale values at particular points (anatomical and time-based) within a given patient's echocardiogram images. For example, a patient's interatrial septum may be used to establish one reference threshold (e.g., black, in the inverted images presented herein), and the middle of the left atrium, prior to injection of bubbles, could be used to establish another reference threshold (e.g., white)—against which thresholds other regions at different points of time could be compared).

In some implementations, machine learning may be applied to distinguish chambers from tissue. In some implementations, only portions of chamber(s) may be delineated. For example, it may only be necessary to delineate atria and portions of one or both of the ventricles that are closest to the atria. In some implementations, delineated structures may be associated with easily identifiable landmarks (e.g., for superposition onto other images at different points in time, to facilitate analyses as described herein)—for example, to points on the exterior heart wall, to portions of the atrial septum, to specific valves that are visible and identifiable at different points in the cardiac cycle, etc.

Regardless of the method employed to identify “regions of interest” (e.g., certain heart chambers), further analysis may be performed within the identified regions of interest. To further depict two analyses, FIGS. 5E-1 and 5E-2 illustrate the ultrasound images of FIGS. 5B-1 and 5B-2, with the regions of interest (as shown, each of the four heart chambers) highlighted.

One analysis that may be advantageous is confirmation of sufficient opacification of the right atrium during a bubble study. Turning to FIG. 5F, ultrasound images of the patient's heart are shown for cycle 02 (a reference, pre-bubble cycle) and cycle 04 (the cycle in which bubbles are first detected in the right atrium). A method may be employed whereby specific subsets of the right atrium are analyzed (e.g., square segments, as illustrated), and colors or grayscale pixel values are compared between different cardiac cycles. The right atrium is shown opacified at cycle 04, and this level of opacification may be quantitatively compared to a threshold value to, in some implementations, confirm sufficient opacification. Moreover, in some implementations, the right atrium is analyzed over multiple cardiac cycles to confirm that it remains opacified for a sufficient number of cycles (e.g., five cycles, in some implementations).

In some implementations, sufficient opacification may be specifically confirmed adjacent to the right atrial septum (e.g., to rule out situations such as those involving patients with Eustachian valves that direct blood from the inferior vena cava (IVC) to the interatrial septum, thereby “washing out” bubbles that are coming from the superior vena cava (SVC)). In some implementations, it may be advantageous to detect “negative contrast” in an otherwise opacified right atrium-which may indicate a significant left-to-right shunt that is sometimes seen with ASDs.

If sufficient opacification (e.g., sufficient level of opacification, for a sufficient number of cardiac cycles) is confirmed, additional analyses may be performed. For example, the left atrium may be analyzed to detect presence of any bubbles within three to five cardiac cycles of when the bubbles first appear on the right side of the heart. FIG. 5G-1 illustrates two cardiac cycles (cycle 08 and cycle 09) during which bubbles are detected in both the left atrium and the left ventricle.

In some implementations, bubbles may be assessed by analysis of grayscale color of individual pixels or clusters or pixels within a region of interest. In some implementations, as depicted in FIG. 5G-2, values associated with clusters of pixels (e.g., the small square segments illustrated) may be averaged and converted to one of a finite number of values (e.g., to facilitate more efficient processing, or to graphically depict or highlight quantities of detected bubbles). For example, clusters of pixels may be converted to one of two binary values (e.g., black or white, as shown in cycle 08 in FIG. 5G-2) or one of multiple different grayscale values (e.g., black, gray, light gray, as shown in cycle 09 in FIG. 5G-2).

In some implementations, color or grayscale values associated with certain numbers of segments within a region of interest or with specific anatomical segments may be employed to “score” a detected number of bubbles, and by extension, a corresponding shunt. For example, in some implementations, a greater number of segments within a region of interest that are determined to include bubbles may be scored higher than a smaller number of bubble-containing segments within the same region of interest. Such a score may then be used to indicate severity of a corresponding shunt. In some implementations, shunts that are associated with high scores may be reviewed for possible surgical repair; shunts that are associated with low scores may be monitored and/or treated with medicine (e.g., blood thinners); and shunts that are associated with intermediate scores may be subjected to additional testing (e.g., a follow-on TEE study, if the present study was a TTE study).

In some implementations, the anatomical location of detected bubbles may influence scoring. For example, detected bubbles very near an atrial wall may be scored lower than bubbles detected at an end of the atrium opposite the atrial septum. As another example, bubbles that are detected in the left ventricle (as shown in FIGS. 5G-1 and 5G-2) may be scored higher than bubbles that are contained within the left atrium. As another example, bubbles that are detected entering the left atrium near the pulmonary veins may be scored in a manner that suggests a transpulmonary shunt. Bubbles that are detected on the left side may be further analyzed relative to time—for example, to differentiate a low-level steady trickle versus a bolus of bubbles appearing spontaneously after a cough or performance of the Valsalva maneuver (with a detected bolus being scored higher, in some implementations, than a low-level trickle). Various scoring methods are contemplated and possible.

Another analysis that may be automated, in some implementations, is confirmation that a Valsalva maneuver was performed property. A Valsalva maneuver (where the patient forces expiration against a closed glottis, to increase intrathoracic pressure and increase venous return upon release of the maneuver) is often performed in conjunction with a bubble study in order to increase right atrial pressure—so as to overcome some of the natural resistance, when a shunt is present, to the passage of blood from right atrium to left atrium (given the inherently higher pressure in the left atrium, without performance of a Valsalva maneuver). Performance of such a maneuver can illuminate shunts that might not otherwise be detectible; such illumination, however, can be dependent on the quality of the maneuver—which ideally (depending on how flexible the patient's septum is) should result in a visible right-to-left shift in the interatrial septum (or visible decrease in left atrium volume). Thus, it can be advantageous to confirm proper performance of the maneuver. In some implementations, coughing during specific periods of a bubble study may have a similar effect as performing a Valsalva maneuver; and certain techniques described herein for confirming proper execution of a Valsalva maneuver may also be employed to confirm desired effect of coughing. Other methods of increasing right-side pressure may also be employed and analyzed.

In some implementations, a method to confirm proper performance of a Valsalva maneuver can analyze right atrium volume changes during performance of the maneuver, relative to a baseline; and confirm either a changed volume of the right atrium or a detectible shift in the interatrial septum. One such exemplary method is now illustrated and described with reference to FIGS. 6A-1, 6A-2, 6A-3; 6B-1, 6B-2, 6B-3; and 6C.

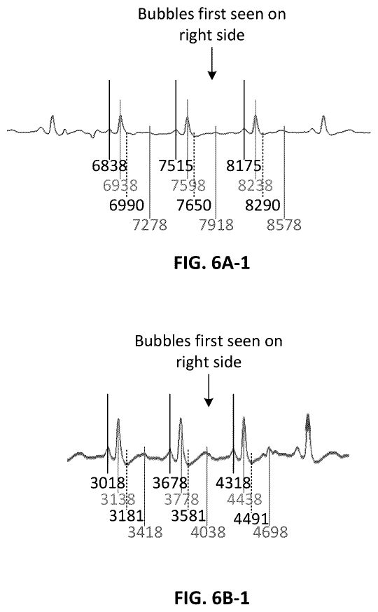

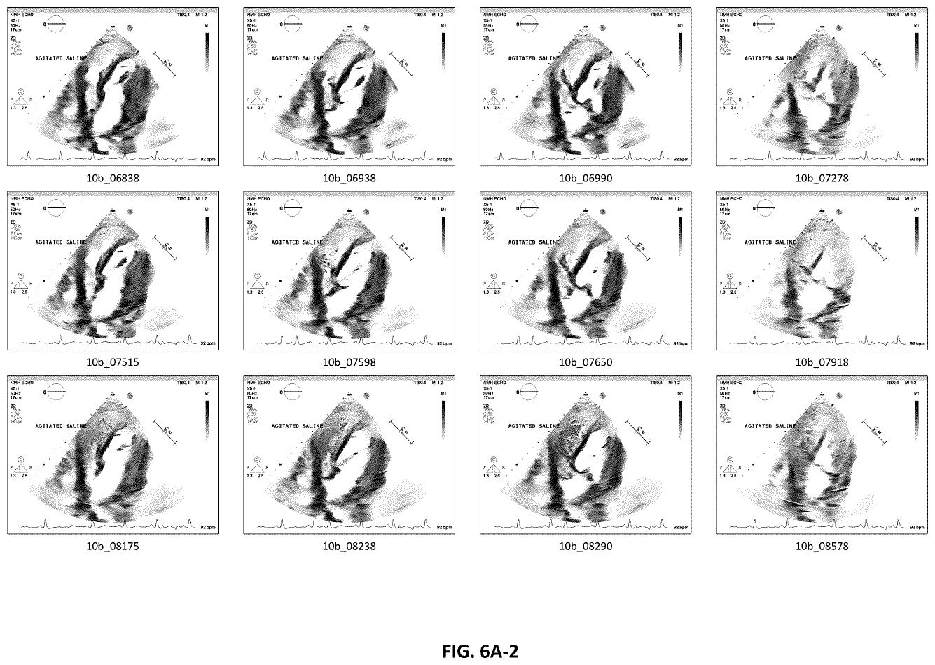

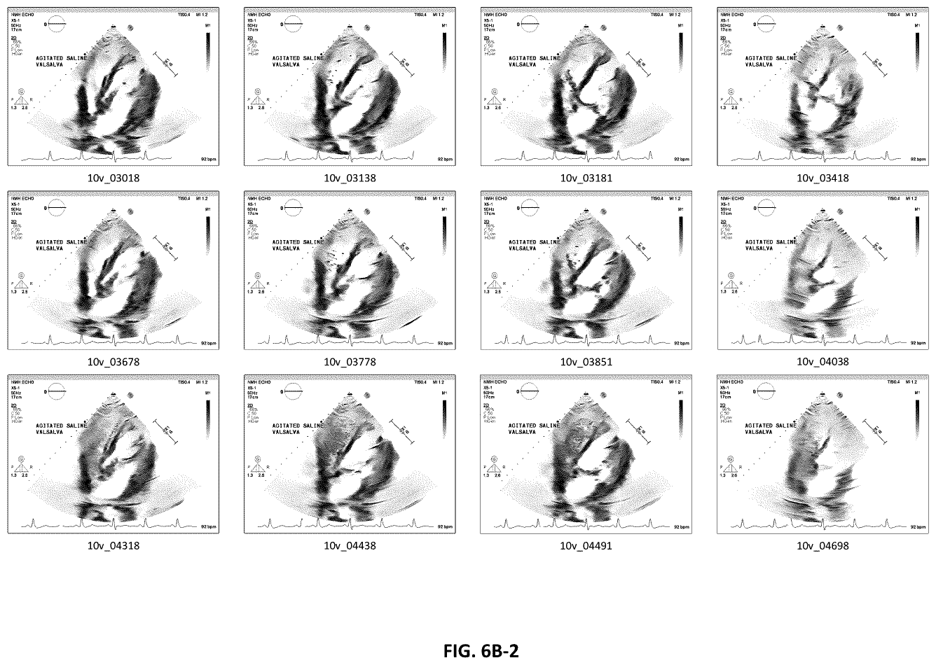

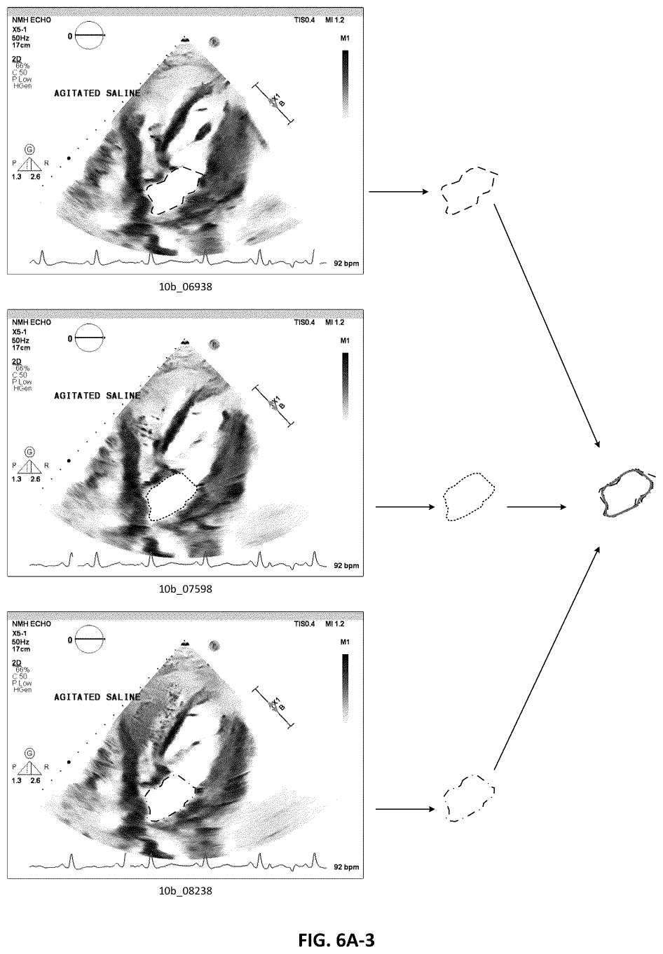

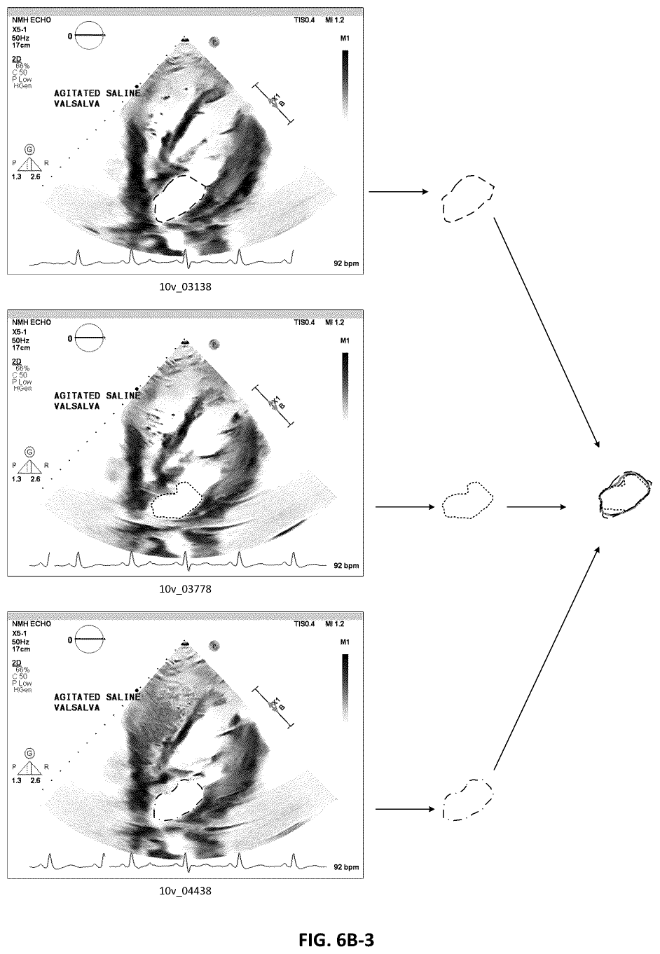

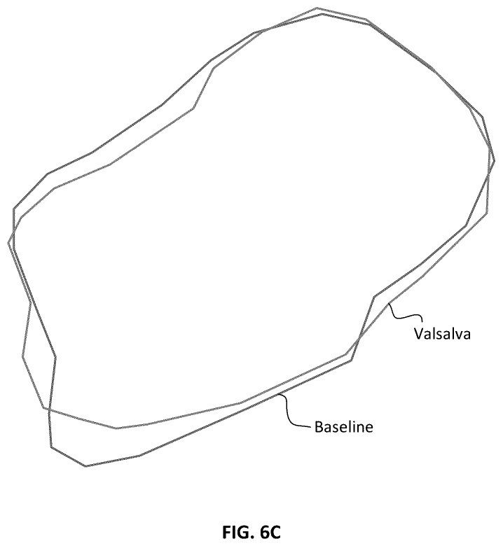

FIG. 6A-1 illustrates a baseline cardiac waveform snapshot for a patient who underwent a bubble study. In the waveform shown, bubbles were injected and detected; but the patient did not perform a Valsalva maneuver. FIG. 6A-2 illustrates corresponding echocardiogram images. Time points are correlated between the images of FIG. 6A-2 and the waveform of FIG. 6A-1. FIG. 6A-3 depicts an analysis of three specific images from the same phase of the patient's cardiac cycle. In particular, FIG. 6A-3 depicts identification of the boundary of the left atrium in each of the images. Further depicted is extraction of the identified boundary from each image and averaging of that boundary to determine an average baseline boundary.

A similar process is depicted for detecting a boundary of the left atrium during a Valsalva maneuver—FIG. 6B-1 illustrates a cardiac waveform snapshot for a patient who underwent a bubble study (while performing a Valsalva maneuver); FIG. 6B-2 illustrates corresponding echocardiogram images (again, correlated to the waveform by the illustrated time points). FIG. 6B-3 depicts an analysis of three specific images from the same phase of the patient's cardiac cycle to determine an average boundary for the left atrium during the Valsalva maneuver. FIG. 6C illustrates a comparison of the two boundaries—which comparison may be further analyzed to determine whether an expected shift in the interatrial septum or another portion of an atrial wall is seen or an expected decrease in left atrium volume. If not, real time feedback may be provided, such that the procedure may be repeated.

Described and illustrated are methods to identify boundaries of the left atrium. But similar techniques could be applied to analyze portions of the atrial septum, or other parameters that can analyzed over time to confirm proper execution of the Valsalva maneuver. For example, ECG data may be analyzed to help confirm proper execution of the Valsalva maneuver (for example, by analyzing the time of the P wave, the amplitude of the P wave, the RR interval, the PR time, the QTc interval, T wave amplitude, T/R amplitude, etc.—parameters that may change during or immediately after a Valsalva maneuver).

Automated image processing, as described herein, may be performed on one or a plurality of machines or devices. For example, portions of methods described here may be performed within onboard computing resources of an ultrasound machine itself; and certain information about the images (e.g., quality and duration of opacification or detection of bubbles; presence of detected bubbles on the left side of the heart; a score associated with such bubble detection; confirmation that a Valsalva or other maneuver resulted in a detectible shift of the interatrial septum; etc.) may be available in real time or nearly in real time. As another example, images may be captured by a bedside ultrasound machine that is proximate a patient undergoing a test, and video and image media may be exported to a supplemental computing device that is also proximate the patient (e.g., a laptop of tablet computing device used by an ultrasound clinician). Certain aspects of the image processing described herein may be performed on that computing device and available with only a short delay (e.g., delay associated with some processing time and the time necessary to transfer media to the supplemental computing device). As another example, media may be captured by an ultrasound machine and transferred via a network to a remote computing device for analysis—either in real time, or for later post-processing. Implementations in which real time processing is possible may be advantageous; as it may be able to identify deficiencies in a procedure aspect while the patient is still present and while the procedure can be easily repeated—for example, a failed or insufficient Valsalva maneuver, or poor or limited-duration opacification on the left side.

### Artificially Intelligent Software (OrbisIQ) for PFO Characterization

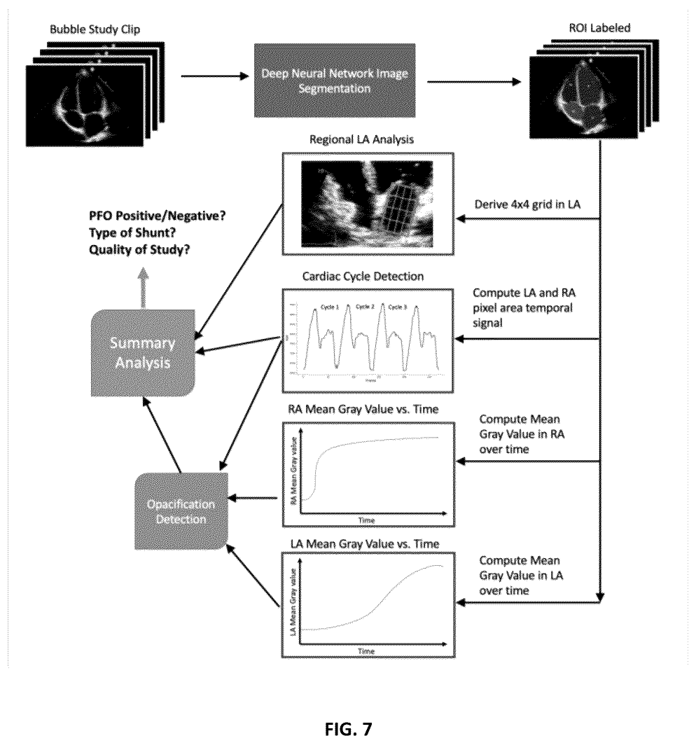

In some implementations, an AI software tool (e.g., “OrbisIQ”) uses TTE or TEE data to determine a source of a shunt (ASD, PFO or PAVM) and estimate the risk of a PFO/ASD allowing embolization of a thrombus to support clinical decision making on the need for closure. The architecture may be as depicted in FIG. 7, with two primary components: 1) A deep neural network image segmentation model that automatically segments the RA and LA chambers and the interatrial septum of the heart from the background in an apical 4-chamber (AP4) TTE clips, and 2) a set of rule-based image analysis algorithms that translate regional image gray values to shunt characterization and left atrium analysis focused on fluid dynamics.

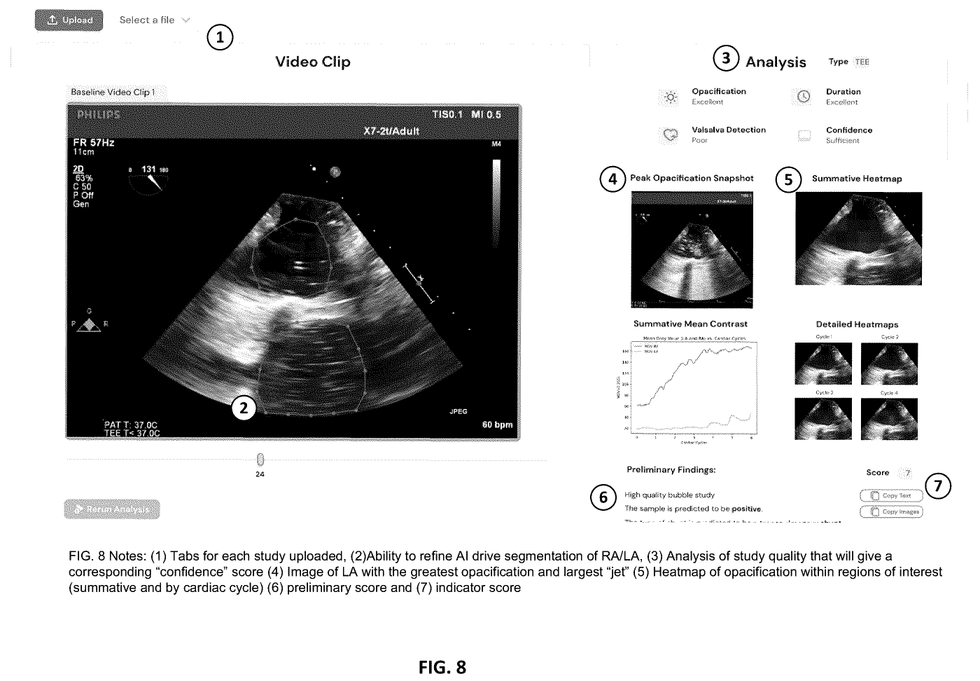

The deep neural network image segmentation model depicted in FIG. 7 was developed with n=1162 TTE human cardiac echo images (non-bubble studies). Each echo image was annotated for the RA, LA, and the interatrial septum in a pixel-wise labels using the ITK-Snap tool. From the n=1162 images, 15% or n=175 images were held out as the validation set for model selection. The training dataset was further augmented with images perturbed with random variations in size, position, and rotation for model generalization. A DeepLabV3+ model architecture2 with a ResNet183 backbone was employed that combines multi-resolution features for improved scale-invariant performance. The model was trained with the DICE loss, initial learning rate of 1e-4, and batch size of 16. The resulting segmented LA and RA regions are represented with polygons to enable user edits as illustrated in an exemplary software tool screen shot in FIG. 8 (see item 2).

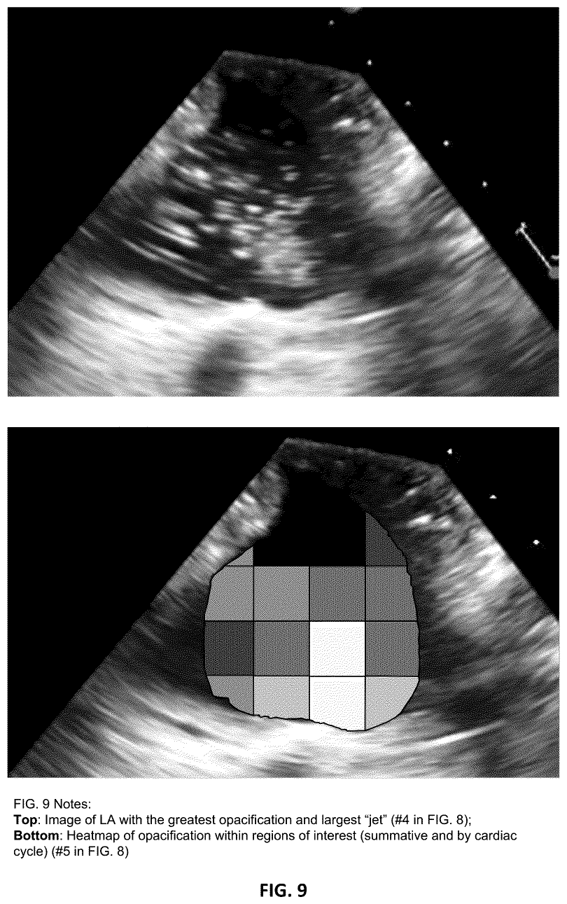

In some implementations, approach to shunt characterization and left atrium analysis may be focused on fluid dynamics. The predicted LA and RA segmentations may be used to compute mean gray value over time for automated cardiac cycle detection and opacification detection of RA (summative) and LA by regions of interest. For cardiac cycle detection, the total number of pixels of both LA and RA regions may be computed for each frame, creating a 1-dimensional temporal signal. A peak-finding algorithm may be used to detect the peaks of the temporal signal, where the peaks represent the end-systole (ES) instance of the cardiac cycle. Peak opacification in both LA and RA may be detected by applying peak finding algorithms to the smoothed mean gray values, with a snapshot of peak opacification in LA shown in item 4 in FIG. 8. For regional LA analysis (see item 5 in FIG. 8) the LA region may be automatically split into a 4×4 grid with individual mean gray values for each cell of the grid calculated over time. Together, these outputs can be used to predict if the study is positive for a PFO, as indicated by opacification of the left atrium within 3 cardiac cycles and the location of the first microbubbles (near the septum or pulmonary veins), and/or positive for a TPS, as indicated by opacification of the left atrium beyond 3 cardiac cycles and location of the first microbubbles as seen in item 6 in FIG. 8. The grid of the LA may also provide insights into shunt severity. OrbisIQ also includes functionality for automated quality control features as seen in item 3 in FIG. 8. Quality of opacification may be derived based on the relative gray values between non-opacified and opacified states. Duration of opacification may be determined by having at least 4 cardiac cycles with sufficient opacification. OrbisIQ may also determine whether an effective Valsalva maneuver was present by modeling the change in curvature of the interatrial septum and detecting for sudden increase of curvature, changes in LA and RA areas, heart rate variability and any changes in opacification. Finally, an overall confidence score on the study may be derived based on these factors. The implementation depicted was trained on 60 historical human SOC bubble studies and 114 Biosimulation echos from ex vivo swine hearts to verify PFO/ASD or TPS presence. The detected cardiac cycles and the mean gray value curves were used to develop the output shown in FIG. 9.

PFO pathogenicity may be determined by the degree of and time-course of opacification of the LA from microbubbles during a bubble study, with greater opacification and a “microbubble bolus” indicative of a more severe PFO.

In some implementations, model training may be based on multiple inputs, including, for example, ex vivo cadaveric human and porcine training samples with SOC contrast injections into healthy hearts, naturally occurring ASD/PFOs, and various sizes of artificially created PFOs all with multiple injections and varying RA pressures to simulate maneuvers; contrast levels in the LA and RA regions of interest over time; and with human bubble study echos for diagnoses of PFO or TPS, and severity of shunt.

Further Refinement to Account for Right Atrial Pressure Changes and Known Shunt Sizes with a Novel Ex Vivo Human and Swine Cadaver Heart System

A database may be created of echocardiograms to further characterize the effect of right atrial pressure changes on shunt characteristics and left atria analysis. This database may be used to further refine the maneuver detection algorithm by evaluating ranges of simulated maneuvers as well as to better characterize how fluid dynamics and pressures influence the same shunt in the left atrium analysis (e.g., to simulate right atrial pressure changes (simulated maneuvers like Valsalva or cough) typically done during bubble studies). Further images may be analyzed of cadaveric heart systems.

Transfer learning is a deep learning method, intended to take a trained model and apply it to a related, secondary task for rapid testing and improved performance of an AI tool. The machine learning model may be fine-tuned with TEE and PFO size data collected in the porcine and human cadaver models. Given the grossly similar anatomy within this US viewing window, the representation learned from the software deep neural network model may translate to the new image domain. The output may be similarly derived, including ROI identification and LA and RA opacification versus time. A secondary task may be additional post-processing of the contrast analysis (peak opacification and opacification by regions of interest in the LA), using the weighted predictive observations found to be indicative of PFO size.

Further Refinement of OrbisIQ to Classify Shunt Type and Size Through Acquisition of Clinical Echocardiograms that have been Previously Assessed and Graded by Cardiologists

Further AI model implementation and training may be completed with additional bubble studies (e.g., bubble study echocardiogram videos split between TEE and TTE and containing apical 4-chamber clips and split between the following categories: 1) negative for right to left shunt, 2) TPS, 3) large shunt that was ultimately closed with no known stroke recurrence, 4) medium ASD or PFO, and 5) small ASD or PFO. The categorization of the bubble studies may be defined and confirmed by two independent cardiologists. To classify among the shunt types, training may be based on a state-of-the-art deep learning-based video classifier called ViViT2, which may be trained to classify between 1) Negative for Right to Left Shunt, 2) TPS, or 3) ASD or PFO. Due to the requirement to learn long-term association of spatiotemporal features, a ViViT with a transformer architecture may be applied to learn long-distance dependencies. This model may be trained with additional data for model testing. Second, an additional classification head may be added to ViViT to predict the size of the shunt for a positive ASD/PFO prediction and training of this classification head may proceed on the labeled positive ASD/PFO samples. For both models, the feature extractor may be pre-trained using self-supervised contrastive learning on large open-source dataset and fine-tuned on the specific tasks as defined in the two-step approach. The model may be implemented and trained with PyTorch22 using Amazon Web Services (AWS).

Prospective Validatation of OrbisIQ by Processing Additional Echocardiograms with and without OrbisIQ and Compare Type and Size of Shunt as Diagnosed by OrbisIQ to Traditional Diagnosis Methods

In a final phase, OrbisIQ's ability to successfully categorizes type and severity of shunt with a sensitivity and specificity of at least 80% may be demonstrated. OrbisIQ may be evaluated with a new set of TEE and TTE clinical cardiac bubble studies, and its automation capabilities assessed statistically. The automated characterization of shunt type (atrial or TPS), shunt severity (negative, small, medium, and large), and whether a maneuver was detected may be scored against the ground truth, which may be generated and verified by the review and agreement of two echocardiographers. A primary endpoint may be sensitivity and specificity of the determination of positive or negative shunt, and secondary endpoints may include: 1) for positive shunts, distinction between an arterial shunt or TPS, and 2) for positive shunts, determination of shunt severity: severe (large) or not severe as determined using the fluid dynamic analysis of the LA and the ground truth of shunts that went on to be closed. Success may be predicated on statistical testing of the co-primary sensitivity endpoint, which may be performed before testing the co-primary specificity endpoint.

While several implementations have been described with reference to exemplary aspects, it will be understood by those skilled in the art that various changes may be made and equivalents may be substituted for elements thereof without departing from the contemplated scope. For example, described herein are methods for characterizing cardiac shunts. The methods could easily be adapted, however, to identify shunts or defects outside of the heart. For example, multiple bubble studies with progressively larger bubbles may be employed to detect and assess pulmonary shunts, aneurysms, plaque buildup or other narrowing of vessels, spasm or constriction of vessel, or other conditions. Techniques described herein could be employed with contrast agents other than bubbles—for example, with Perflutren Protein-Type A Microspheres or other contrast agents that may enhance contrast and improve delineation of left ventricular endocardial borders. References are made to TTE; but techniques described here could be applied in the context of TEE procedures as well. Automated image analysis could be applied to calculate hemodynamics in addition to detecting shunts. In addition, many modifications may be made to adapt a particular situation or material to the teachings provided herein without departing from the essential scope thereof. Therefore, it is intended that the scope not be limited to the particular aspects disclosed but include all aspects falling within the scope of the appended claims.

What is claimed is:

1. A method comprising:
intravenously directing first bubbles into a patient's venous circulatory system and to a right side of the patient's heart, the first bubbles having sizes that fall within a first range;
monitoring the patient's heart, with ultrasound imaging, to detect presence of the first bubbles on a left side of the patient's heart;
upon detecting the first bubbles on the left side of the patient's heart, intravenously directing second bubbles into the patient's venous circulatory system and to the right side of the patient's heart, the second bubbles having sizes that fall within a second range, the second range having sizes that are greater than sizes in the first range;
monitoring the patient's heart, with ultrasound imaging, to detect presence of the second bubbles on a left side of the patient's heart; and
upon detecting the second bubbles on the left side of the patient's heart, intravenously directing third bubbles into the patient's venous circulatory system and to the right side of the patient's heart, the third bubbles having sizes that fall within a third range, the third range having sizes that are greater than sizes in the second range;
upon detecting the third bubbles on the left side of the patient's heart, initiating treatment to minimize risk of stroke in the patient.

2. The method of claim 1, wherein intravenously directing first bubbles comprises injecting an agitated solution of saline or dextrose into a median cubital vein of the patient.

3. The method of claim 1, wherein monitoring the patient's heart with ultrasound imaging comprises conducting either a transthoracic echocardiogram (TTE) or a transesophageal echocardiogram (TEE).

4. The method of claim 1, wherein initiating treatment comprises administering a blood thinner to the patient.

5. The method of claim 1, wherein initiating treatment comprises surgically closing a shunt in the patient's heart using a catheter-delivered percutaneous closure device.

6. The method of claim 1, wherein the first range comprises bubbles having diameters approximately between 8 um and 15 um.

7. The method of claim 6, wherein the second range comprises bubbles having diameters approximately between 15 um and 25 um.

8. The method of claim 7, wherein the third range comprises bubbles having diameters approximately between 25 um and 35 um.

9. A method comprising:
intravenously directing first bubbles into a patient's venous circulatory system and to a right side of the patient's heart, the first bubbles having sizes that fall within a first range;
monitoring the patient's heart, with ultrasound imaging, to detect presence of the first bubbles on a left side of the patient's heart;
upon detecting the first bubbles on the left side of the patient's heart, intravenously directing second bubbles into the patient's venous circulatory system and to the right side of the patient's heart, the second bubbles having sizes that fall within a second range, the second range having sizes that are different than sizes in the first range;
upon detecting the second bubbles on the left side of the patient's heart, initiating treatment to minimize risk of stroke in the patient.

10. The method of claim 9, wherein initiating treatment comprises administering a blood thinner to the patient.

11. The method of claim 9, wherein initiating treatment comprises surgically closing a shunt in the patient's heart using a catheter-delivered percutaneous closure device.

12. The method of claim 9, further comprising determining a blood flow rate, using ultrasound imaging, by monitoring movement of first bubbles or second bubbles within the patient's heart.
